(19) United States
(12) Patent Application Publication
(10) Pub. No.: US 2022/0322995 A1
(43) Pub. Date: Oct. 13, 2022

(54) CONTACT LENS HAVING INTEGRATED LIGHT SOURCE FOR ELECTRORETINOGRAPHY AND METHOD FOR PREPARING SAME

(71) Applicants: KOREA ADVANCED INSTITUTE OF SCIENCE AND TECHNOLOGY, Daejeon (KR); SEOUL NATIONAL UNIVERSITY HOSPITAL, SEOUL (KR)

(72) Inventors: SE JOON WOO, Gyeonggi-do (KR); Seunghyup YOO, Daejeon (KR); Taehyun KIM, Daejeon (KR); Jinouk SONG, Daejeon (KR)

(21) Appl. No.: 17/639,382

(22) PCT Filed: Dec. 2, 2020
(86) PCT No.: PCT/KR2020/011793

§ 371 Date: Mar. 1, 2022

(30) Foreign Application Priority Data
Sep. 2, 2019 (KR) 10-2019-0108355
Sep. 2, 2020 (KR) 10-2020-0111756

(51) Int. Cl. A61B 5/398; G02C 7/04; A61B 3/10; A61B 3/00

(52) U.S. Cl. CPC A61B 5/398; G02C 7/04; A61B 3/10; A61B 3/0008

(57) ABSTRACT

The present specification relates to a contact lens having an integrated light source for electroretinography and a method for preparing same, the contact lens, having an integrated light source for electroretinography, comprising: a light source; a scattering material for scattering light from the light source; and an electrode for measuring changes in the electroretinogram due to the stimulation from the light source.

### TECHNICAL FIELD

The present invention relates to a contact lens with which an electroretinogram of an eyeball can be examined and a method for manufacturing the same. More particularly, the present invention relates to a contact lens in which a light source and an electrode used for electroretinography are integrated and worn in a form of a contact lens, thereby securing excellent examination accuracy, and a method of manufacturing the same.

[National R&D project supporting the present invention]

[Project Identification Number] N11190165

[Ministry in charge] Ministry of Science and ICT

[Research management Institution] Korea Advanced Institute of Science and Technology

[Research project name] Korea Advanced Institute of Science and Technology's own research project

[Research title] Development of Light Source-Integrated Corneal Contact Lens Electrode for Electroretinogram

[Contribution rate] 1/1

[Organizing institution] Korea Advanced Institute of Science and Technology

[Research period] 2019 Apr. 1˜2019 Dec. 31

### RELATED ART

A certain electric potential (electroretinogram) exists in a retina of an eyeball, and this electric potential is changed by stimulation with light. This change can be measured through electroretinography (ERG).

The electroretinography enables screening and confirmation of retinal diseases such as inherited retinal diseases, inflammatory retinal diseases, diabetic retinopathy, etc. and optic nerve diseases such as glaucoma, etc.

Meanwhile, in electroretinography, the intensity of stimulation with light and the exposure time of light are standardized, and thus a configuration of an examination room to meet this requires an expensive examination equipment cost, an examination space, a skilled examiner, etc.

For example, as illustrated in FIG. 12, a full field electroretinography apparatus 1000 in the related art uses an external light source and a corneal electrode, and a subject of examination is supposed to puts his/her face on a large examination machine for the examination.

The full field electroretinography apparatus 1000 in the related art uses the external light source to measure the electroretinogram and thus enables accurate measurement. However, for the purpose, a darkroom and a large space have to be implemented and also there is a disadvantage that the cooperation of the subject of examination is inevitable. Accordingly, it takes a long waiting time to perform electroretinography in a hospital.

In addition, a large machine installation space is required for the electroretinography. Furthermore, since the electrode and the light source are separated, there are inconveniences such that the subject of examination has to keep looking at the center of the light source during the examination time.

In order to address these demerits, electroretinography that enables automatic examination in a small space with relatively little cooperation of the subject of examination is necessary.

### DISCLOSURE

### Technical Problem

In order to address the problems in the related art described above, the present invention provides an electroretinography apparatus capable of significantly reducing time, place, and manpower in an actual ophthalmic examination room, and stably maintaining the optical relationship between a subject of examination and an light source, thereby increasing the effectiveness of the examination.

In addition, the present invention provides an electroretinography method in which an examination can be simplified, space constraints become much less, and an examination cost can also be significantly reduced by providing a light source and electrode-integrated contact lens electroretinography apparatus.

### Technical Solution

In order to achieve the above-described technical object, in example embodiments of the present invention, there is provided a light source-integrated contact lens for electroretinography including: a light source; a scattering material that scatters light from the light source; and an electrode for measuring a change in electroretinogram due to stimulation from the light source.

In an example embodiment, the light source-integrated contact lens for electroretinography may include: a corneal contact portion that is to be in contact with a corneal surface of an eyeball; and a corneal contact electrode disposed on an inner surface of the corneal contact portion, the corneal contact portion may be a scattering layer containing the scattering material, or the corneal contact portion may include the scattering layer, or the scattering layer may be formed at the corneal contact portion, and the light source-integrated contact lens may include a light source disposed at the scattering layer.

In addition, in an example embodiment, the light source-integrated contact lens for electroretinography may include: a corneal contact portion that is to be in contact with a corneal surface of an eyeball; a corneal contact electrode disposed on an inner surface of the corneal contact portion; a scattering layer disposed on an outer surface of the corneal contact portion; and a light source disposed at the scattering layer.

In addition, in an example embodiment, the scattering layer of the present invention may include scattering particles as the scattering material. The scattering layer may be an elastomer layer in which the scattering particles are dispersed.

In addition, in an example embodiment, the thickness of the elastomer layer of the present invention may be 0.8 mm to 1.5 mm, but is not limited thereto. In addition, the elastomer layer may be made of a polybutylene adipate terephthalate (PBAT) resin or polydimethylsiloxane (PDMS), but is not limited thereto.

In addition, in an example embodiment, the scattering particles of the elastomer layer of the present invention may have an average particle size of 50 nm, and the density of the scattering particles of the elastomer layer may be 1 wt % to 5 wt %, but it is not limited thereto.

In addition, in an example embodiment, the scattering particles of the elastomer layer of the present invention may contain SiO₂ or TiO₂ nanoparticles, but are not limited thereto.

In addition, in an example embodiment, the present invention may further include a cable that connects the light source to an external power source.

In addition, in an example embodiment, the light source of the present invention may be located on the scattering layer or at least a part of the light source may be embedded in the scattering layer.

In addition, in an example embodiment, the light source of the present invention may be disposed at a position corresponding to a center point of the corneal contact portion.

In addition, in an example embodiment, the light source may be in a form in which the light source is mounted on a flexible printed circuit board (FPCB).

In addition, in an example embodiment, the light source of the present invention may be an LED element, and the LED element may be included singly or in plurality.

In addition, in an example embodiment, in a case where the LED element of the present invention is included singly, the LED element may be a single white LED element.

In addition, in an example embodiment, a current flowing through the single white LED light source may be 7.5×10⁻⁴ mA to 0.0185 mA, but is not limited thereto.

In addition, in an example embodiment, in a case where the LED elements of the present invention are included in plurality, the LED elements may be three LED elements of red, blue, and green, or may include a plurality of white LED elements.

In addition, in an example embodiment, an amount of light from the light source may be 5.4×10⁻⁶ cd to 1.2×10⁻¹ cd, but is not limited thereto.

In addition, in an example embodiment, a luminous intensity of light scattered from the scattering layer may be 0.03 cd·s/m² to 3.0 cd·s/m², but is not limited thereto.

In addition, in an example embodiment, the corneal contact electrode of the present invention is, for example, in the shape of a ring, and the size of the diameter of the ring may be larger than the diameter of an iris.

In addition, in an example embodiment, the contact lens may be used to measure not only an electroretinogram of humans or but also an electroretinogram of animals other than humans.

Meanwhile, in example embodiments of the present invention, as a method for manufacturing the contact lens for electroretinography described above, there is provided a method of manufacturing a light source-integrated contact lens for electroretinography, including: providing a solution containing a scattering material; and providing a light source to the solution and curing the solution.

In an example embodiment, the method may further include: providing a corneal contact portion that is to be in contact with a corneal surface of an eyeball, wherein the solution containing the scattering material and the light source may be provided to an outer surface of the corneal contact portion and be cured. Accordingly, a scattering layer in which the light source is joined to the outer surface of the corneal contact portion that is to be in contact with the corneal surface of the eyeball may be formed.

In addition, in an example embodiment, the solution containing the scattering material and the light source may be provided to a mold capable of manufacturing a lens shape and be cured.

### Advantageous Effects

According to the example embodiments of the present invention, the light source and the electrode are integrated in the form of a contact lens which is able to be worn on the eye, so that the retina can be stimulated with light without the need for looking at a target of the light source.

In addition, uniform light stimulation can be transmitted to the retina by the scattering material or the scattering layer of the contact lens, so that excellent examination accuracy can be secured. Accordingly, there is no need to install a separate light source. In addition, since the internal light source is used, an examination can be performed in a small space without preparing a darkroom by blocking the eyes of a subject of examination from the outside.

In addition, the cooperation of the subject of examination is not inevitable and the examination can be performed automatically.

### DESCRIPTION OF DRAWINGS

FIG. 1 is a conceptual view schematically illustrating a cross-sectional perspective view of a contact lens for electroretinography in an example embodiment of the present invention.

FIG. 2 is an enlarged view schematically illustrating a portion on which a light source is mounted according to an example embodiment of the present invention.

FIG. 3 is a schematic view schematically illustrating a light source being embedded in a scattering layer according to an example embodiment of the present invention.

FIG. 4 is an enlarged view schematically illustrating a corneal contact portion and a scattering layer according to an example embodiment of the present invention.

FIG. 5A schematically illustrates a case where a single LED element is used according to an example embodiment of the present invention, and FIG. 5B is a schematic view schematically illustrating an operation mechanism according thereto.

FIG. 6A schematically illustrates a case where three LED elements are used according to an example embodiment, and FIG. 6B is a schematic view schematically illustrating an operation mechanism according thereto.

FIG. 7 is a schematic view schematically illustrating a comparison of a size of the corneal contact electrode with a size of an iris of an eyeball in an example embodiment of the present invention.

FIG. 8A is a graph showing a distribution of each wavelength of a white LED element according to an example embodiment of the present invention, and FIG. 8B is a graph showing a luminous intensity according to a change in a voltage of the white LED element.

FIG. 9 is a graph showing an evaluation result of a scattering layer manufactured according to an example embodiment of the present invention.

FIGS. 10A and 10B each show an electric current and a luminous intensity according to a voltage applied to an LED element using a scattering layer manufactured according to an example embodiment of the present invention.

FIGS. 11A and 11B each show a result of measuring a luminous intensity of the contact lens according to an example embodiment of the present invention by varying a current and an irradiation time.

FIG. 12 schematically illustrates a full field electroretinography apparatus in the related art.

### DESCRIPTION OF REFERENCE NUMERALS

1 Contact lens for electroretinography

10 Corneal contact portion

11 Corneal contact electrode

20 Scattering layer

21 Scattering particles

30 Light sources

30a Red LED element

30b Blue LED element

30c Green LED element

31 Divergent light

31a Red light

31b Blue light

31c Green light

32, 32a Scattered light or scattered white light

50 Iris

D Diameter of corneal contact electrode

d Diameter of iris

### MODE FOR INVENTION

Hereinafter, a contact lens for electroretinography according to an embodiment of the present invention will be described with reference to the accompanying drawings, through preferred example embodiments of the present invention.

In various example embodiments, like constituent elements having the same configuration will be described in a representative example embodiment using like reference numerals, and in other example embodiments, only different constituent elements will be described.

In the present disclosure, a contact lens not only includes a contact lens commonly used for vision control such as, for example, a hard contact lens or a soft contact lens, but also includes a contact lens having a shape of a contact lens without a function of the vision control. For example, in a case where a scattering layer itself containing a scattering material is a corneal contact portion, the corneal contact portion may be included in the context of the contact lens even if there is no vision control function in the corneal contact portion.

In the present disclosure, the corneal contact portion is a portion of the contact lens that is to be in contact with the cornea of an eyeball, and is a portion formed concavely to conform to the shape of the cornea of the eyeball.

In the present disclosure, the scattering layer may be the corneal contact portion itself, or may be included in the corneal contact portion, or may be formed on the outer surface of the corneal contact portion.

In the present disclosure, the meaning that a light source is formed at the scattering layer may include that the light source is located on the scattering layer or that at least a part of the light source is embedded in the scattering layer.

In the present disclosure, a light source may include not only a light source such as an LED itself, but also a circuit board combined type such as, for example, a light source in which the LED is mounted on a flexible printed circuit board (FPCB).

Light Source-Integrated Contact Lens for Electroretinography

In example embodiments of the present invention, there is provided a light source-integrated contact lens for electroretinography including: a light source; a scattering material that scatters light from the light source; and an electrode for measuring a change in electroretinogram due to stimulation from the light source.

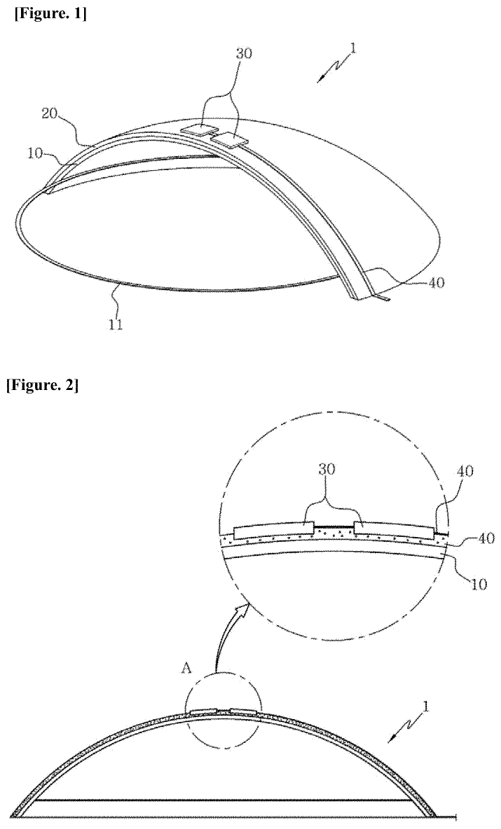

FIG. 1 is a conceptual view schematically illustrating a cross-sectional perspective view of a light source-integrated contact lens 1 for electroretinography in an example embodiment of the present invention.

As illustrated in FIG. 1, the light source-integrated contact lens 1 for electroretinography according to the embodiment of the present invention may include a corneal contact portion 10 that is to be in contact with the corneal surface of an eyeball, a corneal contact electrode 11 disposed on an inner surface of the corneal contact portion 10, and a scattering layer 20 and a light source 30 disposed on an outer surface of the corneal contact portion 10.

In the example embodiment, it is described that the scattering layer is disposed on the outer surface of the corneal contact portion 10. However, in other example embodiments, the corneal contact portion 10 itself may be the scattering layer, or at least a part of the corneal contact portion 10 may include the scattering layer.

Additionally, the light source-integrated contact lens 1 for electroretinography according to the example embodiment of the present invention may further include a cable 40 that supplies power from an external power source (not illustrated) to the light source 30.

Specifically, the corneal contact portion 10 may be provided with the corneal contact electrode 11 on the lenticular concave surface that is to come into contact with the corneal surface of the eyeball, and the scattering layer 20 may be disposed on the opposite side of the concave surface of the corneal contact electrode 11.

The light source 30 may be an LED element, for example, an LED element that emits light having a white light wavelength. In addition, the light source 30 has to have a sufficiently thin thickness. Furthermore, for a smooth examination and research, it is preferable that the light source 30 has a resolution of a very low luminance difference from 0.01 cd/m² to 5 cd/m², enables fine brightness adjustment, and is able to adjust a light emission time in microseconds (ms).

The contact lens 1 of the present invention is useful for measuring not only the electroretinogram of humans, but also the electroretinogram of various animals other than humans. Examples of animals other than humans include mice, rats, rabbits, dogs, cats, pigs, and primates, but are not limited thereto.

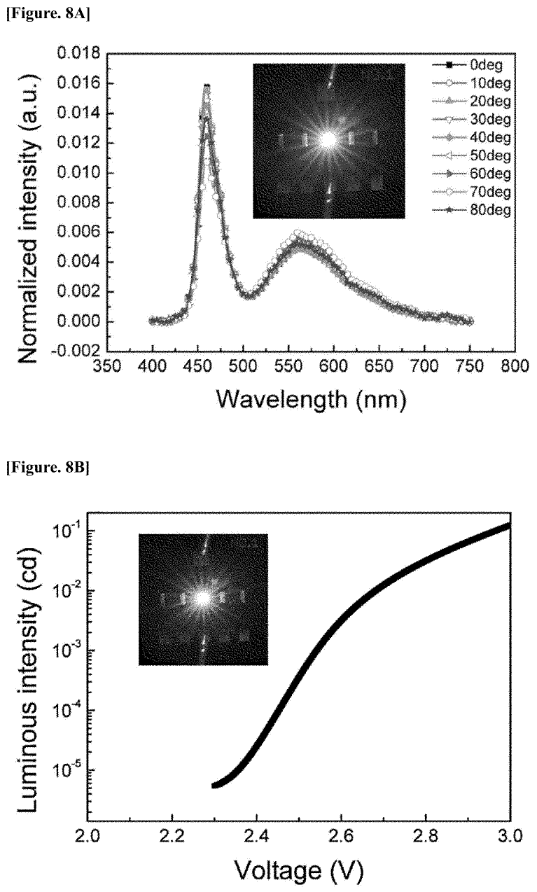

In a case where a white LED element is used as the light source 30 according to the example embodiment of the present invention, the characteristics thereof are shown in FIGS. 8A and 8B

As shown in FIGS. 8A and 8B, for example, a white LED element having a thickness of 0.25 mm shows a maximum brightness of 0.2 cd, and the color coordinates (CIE) are measured as (0.28, 0.27). In addition, when the luminous intensity of the light source 30 using such a white LED element is measured in units of 1 mV, the brightness can be measured in a range of 5.4×10⁻⁶ cd (2.3 V) to 1.2×10⁻¹ cd (3 V).

Therefore, the white LED element light source can measure the LED brightness in units of 1 mV, and since the luminous intensity increases by a multiple of less than 0.3 log unit when converted in units of 1 mV, it is confirmed that the white LED element light source has an appropriate resolution for ERG examination.

Meanwhile, the scattering layer 20 may contain scattering particles, and detailed explanation in this regards will be described later.

FIG. 2 is an enlarged view schematically illustrating a portion on which the light source 30 is mounted according to an example embodiment of the present invention, and FIG. 3 is a schematic view schematically illustrating the light source 30 being embedded in the scattering layer 20 according to an example embodiment of the present invention.

As illustrated in FIGS. 2 and 3, the light source 30 may be located on the scattering layer 20, or a part of the light source 30 may be embedded and disposed in the scattering layer 20.

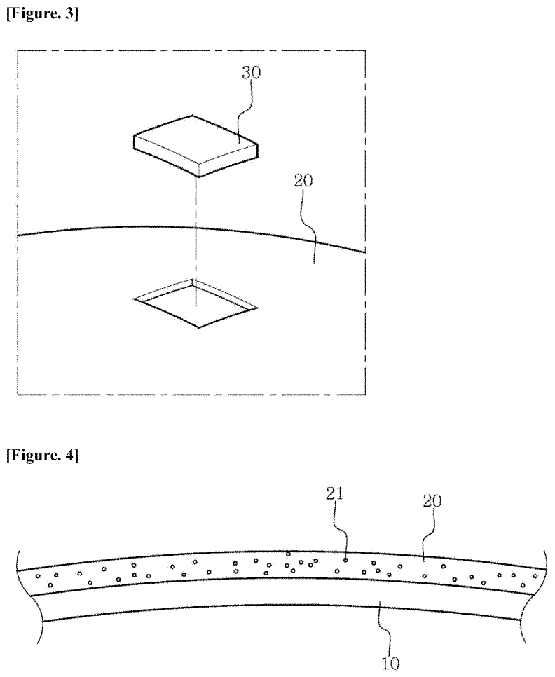

FIG. 4 is an enlarged view schematically illustrating the corneal contact portion 10 and the scattering layer 20 according to an example embodiment of the present invention.

As illustrated in FIG. 4, the scattering layer 20 may include scattering particles 21 dispersed therein. For example, the scattering layer 20 of the present invention may be configured such that the scattering particles 21 are dispersed in an elastomer layer.

The elastomer layer 20 to which the LED element 30 is joined has to be made of a material harmless to the human body, and has to have a shape that is easy to join with the corneal contact portion 10. The thickness of the elastomer layer 20 may be, for example, 2 mm or less, 1.9 mm or less, 1.8 mm or less, 1.7 mm or less, 1.6 mm or less, 1.5 mm or less, 1.4 mm or less, 1.3 mm or less, 1.2 mm or less, 1.1 mm or less, or 1.0 mm or less, or may be 0.1 mm or more, 0.2 mm or more, 0.3 mm or more, 0.4 mm or more, 0.5 mm or more, 0.6 mm or more, 0.7 mm or more, 0.8 mm or more, 0.9 mm or more, or 1.0 mm or more, and for example, may be 0.8 mm to 1.5 mm, but is not limited thereto. The elastomer layer 20 may be made of, for example, a polybutylene adipate terephthalate (PBAT) resin or polydimethylsiloxane (PDMS), but is not limited thereto.

Meanwhile, in order to accurately measure the electroretinogram, the retina has to be irradiated with uniform light. Since the distance between the light source 30 and the retina is very close as the light source 30 is integrated and a point light source, for example, an LED element is used as the light source 30, a scattering layer is necessary to resolve this issue.

In addition, when the concentration of the scattering particles 21 is increased too much for a sufficient scattering phenomenon, a problem that the amount of light ultimately transmitted is reduced may be incurred. Therefore, it is necessary to maintain the amount of light transmitted to a certain level or higher while sufficiently increasing the uniformity.

For example, the scattering particles 21 of the elastomer layer 20 may include SiO₂ or TiO₂ nanoparticles, and the average particle size may be a nano size, that is, 100 nm or less, 90 nm or less, 80 nm or less, 70 nm or less, 60 nm or less, or 50 nm or less, and 10 nm or more, 20 nm or more, 30 nm or more, 40 nm or more, 50 nm or more, and may be, for example, 20 nm to 80 nm, or 40 to 60 nm or 50 nm, but is not limited thereto. For reference, the average particle size may be confirmed through, for example, an SEM photograph.

The density (concentration) thereof may be, for example, 10 wt % or less, 9 wt % or less, 8 wt % or less, 7 wt % or less, 6 wt % or less, 5 wt % or less, and 1 wt % or more, 2 wt % or more, 3 wt % or more, 4 wt % or more, or 5 wt % or more, and may be, for example, 1 wt % to 5 wt %, but is not limited thereto.

These scattering particles 21 may be manufactured by a method of removing (degassing) gases that may be contained in the elastomer layer using a vacuum pump after being applied to the elastomer layer, and curing the resultant in a lens shape. To this end, the light of the light source 30 may be dispersed only with the contact lens without an additional lens or a dispersion layer, and thereby, the simplification of equipment and a cost reduction may be obtained, and accurate measurement is possible through irradiation of uniform light to the retina.

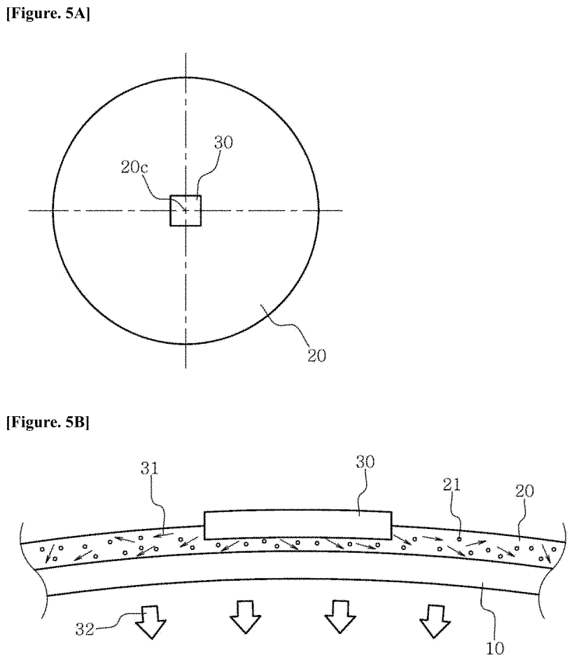

FIG. 5A schematically illustrates a case where, for example, a single LED element is used as the light source 30 according to an example embodiment of the present invention, and FIG. 5B is a schematic view schematically illustrating an operation mechanism according thereto.

As illustrated in FIG. 5A, a single LED element used as the light source 30 is preferably disposed at a position corresponding to a center point 20c of the elastomer layer 20. In addition, as illustrated in FIG. 5b, light 31 is emitted from the light source 30. For example, the single LED element is a white LED element and emits white single light

As such, the divergent light 31 emitted from the light source is scattered by the scattering particles 21 of the scattering layer 20, and finally, scattered light 32 uniformly scattered is emitted throughout the whole inside of the corneal contact portion 10. Therefore, the scattered light 32 reaching the eyeball can be uniformly transmitted.

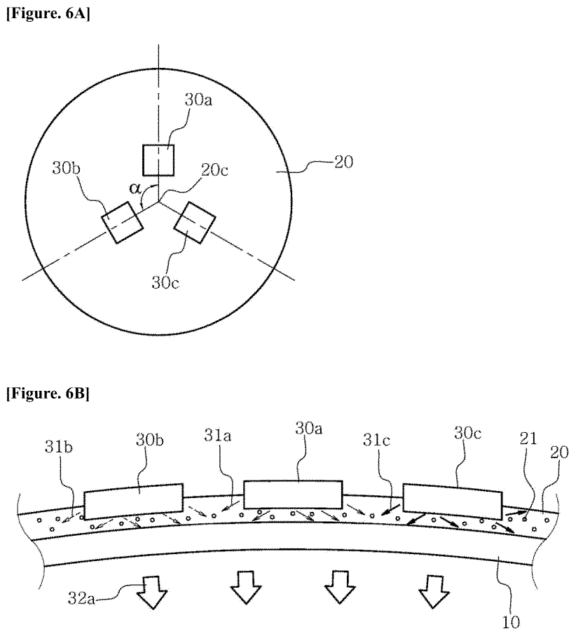

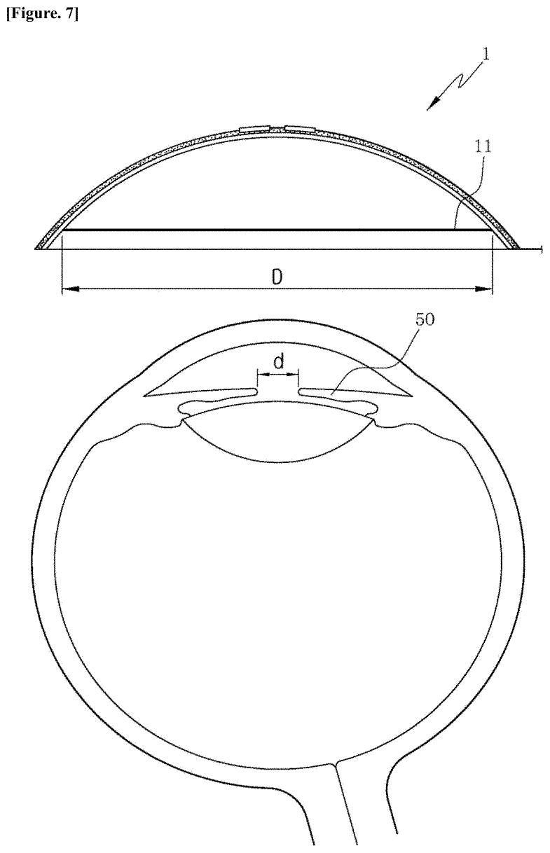

FIG. 6A schematically illustrates a case where, as three LED elements, red, blue, and green LED elements 30a, 30b, and 30c are used according to an example embodiment of the present invention, and FIG. 6B is a schematic view schematically illustrating an operation mechanism according thereto.

As illustrated in FIG. 6A, the three LED elements 30a, 30b, and 30c are preferably arranged at the same distance and angle α around the center point 20c, and even in a case where more than three LED elements are used, it is preferred that the arrangement angles between the LED elements are the same.

In addition, as illustrated in FIG. 6B, the three LED elements 30a, 30b, and 30c respectively emit red single light 31a, blue single light 31b, and green single light 31c, the three single lights 31a, 31b, and 31c emitted are scattered by the scattering particles 21 of the scattering layer 20, and finally white light 32a uniformly scattered is emitted throughout the whole inside of the corneal contact portion 10. Therefore, the white light 32a reaching the eyeball can be uniformly transmitted.

Meanwhile, in the above example embodiments, although the LED elements 30, 30a, 30b, 30c are arranged at or around the center of the scattering layer 20, since uniform white light can be emitted throughout the whole inside of the corneal contact portion 10 through the scattering layer 20, the arrangement position of the LED elements is not limited to the above example embodiments.

In addition, in the above example embodiments, although the three LED elements of red, blue, and green are used in a case where a plurality of LED elements 30, 30a, 30b, and 30c are used, it is possible to use a plurality of white LED elements without differences in color. Therefore, the colors of the LED elements are not limited to the above example embodiments.

In addition, in the above example embodiments, although the LED elements 30, 30a, 30b, and 30c are used, the same effect of the present invention may be expected even in a case where other light sources are used instead of the LED elements.

FIG. 7 is a schematic view schematically illustrating a comparison of a size of the corneal contact electrode 11 with a size of an iris 50 of the eyeball in an example embodiment of the present invention.

As illustrated in FIG. 7, a ring-shaped electrode may be used as the corneal contact electrode 11, and a diameter D of the ring-shaped corneal contact electrode 11 is preferably set to be larger than a diameter d of the iris 50 so that the transmission of light transmitted to the retina of the eyeball is not impeded.

Method of Manufacturing Light Source-Integrated Contact Lens for Electroretinography

Meanwhile, in example embodiments of the present invention, as a method for manufacturing the contact lens for electroretinography described above, there is provided a method for manufacturing a light source-integrated contact lens for electroretinography, including: providing a solution containing a scattering material; and providing a light source to the solution and curing the solution.

Specifically, in an example embodiment, the method for manufacturing a contact lens for electroretinography of the present invention may include: providing a corneal contact portion that is to be in contact with the corneal surface of an eyeball; providing a solution containing a scattering material on the outer surface of the corneal contact portion; and providing a light source to the solution and curing the solution. Accordingly, a scattering layer in which the light source is joined to the outer surface of the corneal contact portion that is to be in contact with the corneal surface of the eyeball can be formed. Here, the corneal contact portion wherein the corneal contact electrode has already been disposed on the inner surface of the corneal contact portion may be used. Alternatively, the corneal contact electrode may be formed on the inner surface of the corneal contact portion after forming the scattering layer to which the light source is joined.

In the example embodiment, since the solution including the scattering material is cured subsequently, a material for curing may be contained. The material for curing is not limited as long as the material can be used for the human body. For example, a precursor of the elastomer and a curing agent may be contained in order for the scattering layer to contain the elastomer as described above.

In an example, in order to produce a scattering layer, for example, 0.9 g and 1.5 g of spherical TiO₂ powder are each dispersed in 10 g of a curing agent, the resultant is mixed in 20 g of a polydimethylsiloxane precursor and then blended using a stirrer, and a degassing is performed using a vacuum pump. Thereafter, a white LED light source is disposed so as to be embedded and curing is carried out.

Meanwhile, in another example embodiment, in order to make the corneal contact portion to be the scattering layer itself, the solution including the scattering material and the light source may be provided to a mold capable of manufacturing a contact lens shape and curing is carried out.

The mold capable of manufacturing a contact lens shape may be divided into, for example, a lower mold region and an upper mold region, and a lens-shaped scattering layer may be made by providing the solution to the gap between the lower mold region and the upper mold region, curing, and then removing the lower mold region and the upper mold region.

Meanwhile, in an example, after mounting the light source on a flexible printed circuit board (FPCB), the light source mounted on the FPCB may be used. A contact lens having a scattering layer to which the light source, which is mounted on the FPCB, is joined may be manufactured, for example, by disposing the light source, in which the light source is mounted on the flexible circuit board (FPCB), in the mold capable of manufacturing a lens shape, and providing and curing the solution containing the scattering material thereto.

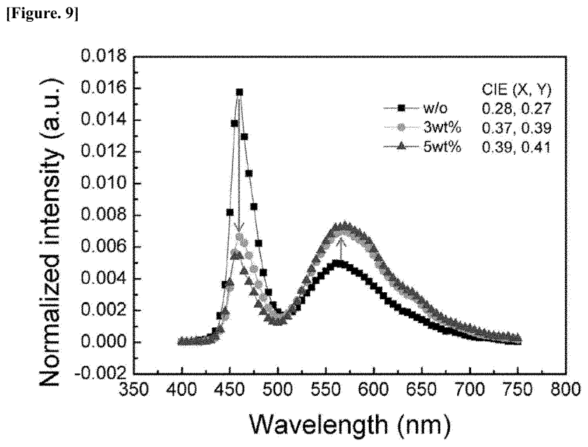

FIG. 9 is a graph showing an evaluation result of the scattering layer produced according to an example embodiment of the present invention.

Compared to the wavelength distribution of the white LED light source of FIG. 8A described above, it can be confirmed that as the wt % of TiO₂ increases, the peak of the blue wavelengths decreases since TiO₂ absorbs blue wavelengths having short wavelengths, and the yellow wavelengths relatively increase, and thus the target color coordinates (CIE) approaches (0.3, 0.3).

That is, it can be confirmed that even if the proportion of TiO₂ increases, a large scattering effect has not been exhibited and it is rather sensitive to thickness. Therefore, assuming that a lens is produced using a 1 mm scattering layer containing 3 wt % of TiO_{2,} it is preferable to use three to four LED light sources to provide stimulation with light.

In addition, in order to match the target color coordinates (CIE) of (0.31, 0.31), it is preferable to use TiO₂ in a ratio reduced to about 1 wt %.

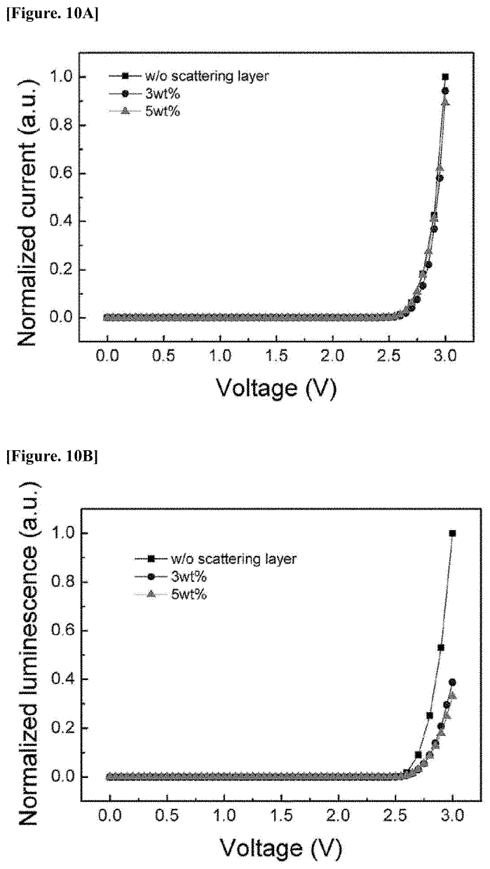

FIGS. 10A and 10B each show an electric current and a luminous intensity according to a voltage applied to an LED using a scattering layer produced according to an example embodiment of the present invention.

As illustrated in FIGS. 10A and 10B, it is confirmed that when the voltage is increased and exceeded 2.5 V, an electric current response occurs, and accordingly the luminous intensity is increased.

Since the luminous intensity of light scattered by the scattering layer 20 of the lens according to an example embodiment of the present invention is preferably at least 0.03 cd·s/m² and at most 3.0 cd·s/m², the electric current flowing through the white LED light source is preferably adjusted to 7.5·10⁻⁴ mA or more and 0.0185 mA or less.

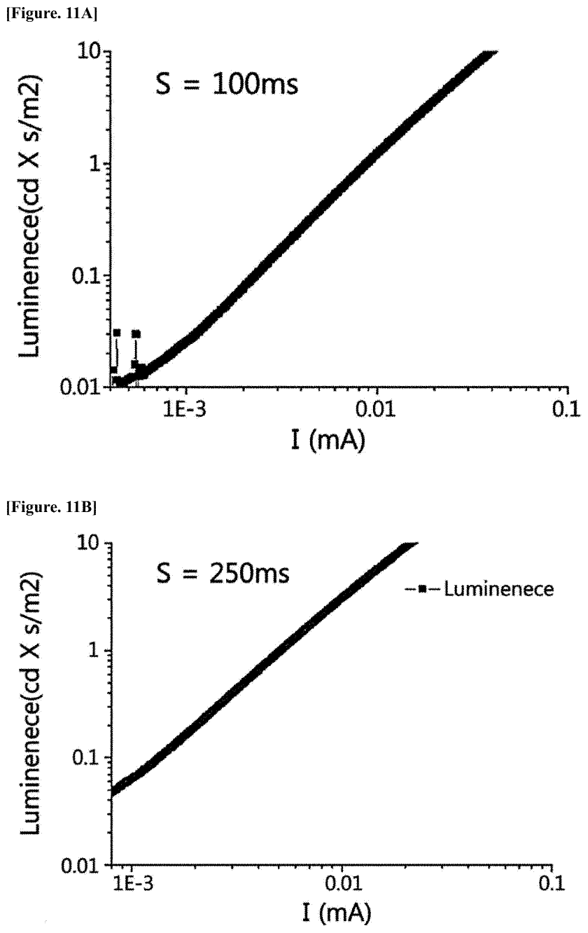

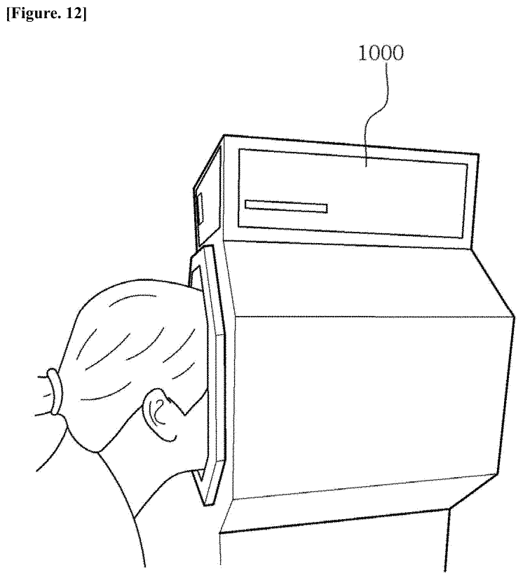

FIGS. 11A and 11B are graphs showing a result of measuring the luminous intensity in the contact lens 1 according to an example embodiment of the present invention by varying an electric current and an irradiation time.

Specifically, in order to measure the amount of light of a single white LED element, the amount of light is measured in a state where the element is inserted into a scattering layer having a thickness of, for example, 0.8 mm. The scattering layer is produced to have the scattering materials concentration of 1 wt %, and the thickness thereof is 0.8 mm.

In the measurement of the luminous intensity, light measured through a photodiode is calculated in cd, and in order to convert this into cd/m², the aperture is reduced using the pr-670 device, thereby focusing on a portion having the strongest light intensity in the scattering layer.

The measured amount of light was inverted to calculate the light of the LED including the scattering layer in cd/m², and in order to convert this into cd·s/m², which is a standard unit of electroretinography, the derived value was multiplied by 100 ms and 250 ms and calculated, respectively.

As shown in FIG. 11A, it is confirmed that in order to satisfy an amount of light necessary for 0.03 ERG, an electric current of 1.13×10⁻³ mA is necessary when irradiating light for 100 ms, and an electric current of 5.17×10⁻⁴ mA is necessary when irradiating light for 250 ms.

In addition, as shown in FIG. 11B, in order to satisfy an amount of light necessary for 3.0 ERG, an electric current of 4.34×10⁻³ mA is necessary when irradiating light for 100 ms, and an electric current of 2.59×10⁻³ mA is necessary when irradiating light for 250 ms.

With reference to the above descriptions, those skilled in the art to which the present invention pertains will be able to understand that the present invention can be implemented in other specific forms without changing the technical spirit or essential features thereof.

Therefore, it should be understood that the example embodiments described so far are illustrative in all respects and are not intended to limit the present invention to the example embodiments, and the scope of the present invention is based on the claims described below rather than the above detailed description. All changes or modifications derived from the meaning and scope of the claims and equivalent concepts should be interpreted as being included in the scope of the present invention.

### INDUSTRIAL APPLICABILITY

The present invention relates to a contact lens with which the electroretinogram of an eyeball can be examined and a method of manufacturing the same, wherein a light source and an electrode used for electroretinography are integrated and worn in a form of a contact lens, thereby securing excellent examination accuracy.

1. A light source-integrated contact lens for electroretinography comprising:
a light source;
a scattering material that scatters light from the light source; and
an electrode for measuring a change in electroretinogram due to stimulation from the light source.

2. The light source-integrated contact lens for electroretinography according to claim 1, comprising
a corneal contact portion that is to be in contact with a corneal surface of an eyeball;
a corneal contact electrode disposed on an inner surface of the corneal contact portion; and
a light source disposed at the scattering layer,
wherein the corneal contact portion is a scattering layer comprising the scattering material, or the corneal contact portion comprises the scattering layer, or the scattering layer is formed at the corneal contact portion.

3. The light source-integrated contact lens for electroretinography according to claim 1, comprising
a corneal contact portion that is to be in contact with a corneal surface of an eyeball;
a corneal contact electrode disposed on an inner surface of the corneal contact portion;
a scattering layer disposed on an outer surface of the corneal contact portion; and
a light source disposed at the scattering layer.

4. The light source-integrated contact lens for electroretinography according to claim 2,
wherein the scattering layer comprises scattering particles as the scattering material.

5. The light source-integrated contact lens for electroretinography according to claim 4,
wherein the scattering layer is an elastomer layer in which the scattering particles are dispersed.

6. The light source-integrated contact lens for electroretinography according to claim 5,
wherein the elastomer layer is made of a polybutylene adipate terephthalate (PBAT) resin or polydimethylsiloxane (PDMS).

7. The light source-integrated contact lens for electroretinography according to claim 5,
wherein the scattering particles of the elastomer layer are SiO₂ or TiO₂ nanoparticles.

8. The light source-integrated contact lens for electroretinography according to claim 1, further comprising:
a cable that connects the light source to an external power source.

9. The light source-integrated contact lens for electroretinography according to claim 2,
wherein the light source is located on the scattering layer or at least a part of the light source is embedded in the scattering layer.

10. The light source-integrated contact lens for electroretinography according to claim 2,
wherein the light source is disposed at a position corresponding to a center point of the corneal contact portion.

11. The light source-integrated contact lens for electroretinography according to claim 1,
wherein the light source has a form in which the light source is mounted on a flexible printed circuit board (FPCB).

12. The light source-integrated contact lens for electroretinography according to claim 1,
wherein the light source is an LED element.

13. The light source-integrated contact lens for electroretinography according to claim 12,
wherein the LED element is included singly or in plurality.

14. The light source-integrated contact lens for electroretinography according to claim 13,
wherein the LED element is a single white LED element.

15. The light source-integrated contact lens for electroretinography according to claim 13,
wherein the LED element is a plurality of white LED elements.

16. The light source-integrated contact lens for electroretinography according to claim 13,
wherein the LED element is included in plurality to have three LED elements of red, blue, and green.

17. The light source-integrated contact lens for electroretinography according to claim 1,
wherein the contact lens is used to measure an electroretinogram of humans or to measure an electroretinogram of animals other than humans.

18. A method of manufacturing the light source-integrated contact lens for electroretinography according to claim 1, the method comprising:
providing a solution comprising a scattering material; and
providing a light source to the solution and curing the solution.

19. The method of manufacturing the light source-integrated contact lens for electroretinography according to claim 18, further comprising:
providing a corneal contact portion that is to be in contact with a corneal surface of an eyeball,
wherein the solution comprising the scattering material and the light source are provided to an outer surface of the corneal contact portion and cured.

20. The method of manufacturing the light source-integrated contact lens for electroretinography according to claim 18,
wherein the solution comprising the scattering material and the light source are provided to a mold capable of manufacturing a lens shape and cured.
